(19) United States
(12) Patent Application Publication
(10) Pub. No.: US 2013/0089631 A1
(43) Pub. Date: Apr. 11, 2013

(54) COMPOSITION CONTAINING COLORED-BEAN EXTRACTS

(71) Applicants: Jin Kwan Kim, Suwon-si (KR); Chae Wook Kim, Yongin-si (KR); Yeon Su Jeong, Yongin-si (KR); Jun Won Yun, Bucheon-si (KR); Kyung Min Lim, Hwaseong-si (KR); Yung Hyup Joo, Yongin-si (KR); Young Ho Park, Seoul (KR); Hyun Jung Shin, Seoul (KR)

(72) Inventors: Jin Kwan Kim, Suwon-si (KR); Chae Wook Kim, Yongin-si (KR); Yeon Su Jeong, Yongin-si (KR); Jun Won Yun, Bucheon-si (KR); Kyung Min Lim, Hwaseong-si (KR); Yung Hyup Joo, Yongin-si (KR); Young Ho Park, Seoul (KR); Hyun Jung Shin, Seoul (KR)

(73) Assignee: AMOREPACIFIC CORPORATION, Seoul (KR)

(21) Appl. No.: 13/704,805

(22) PCT Filed: Jun. 17, 2011
(86) PCT No.: PCT/KR2011/004450

§ 371 Date: Dec. 17, 2012

(30) Foreign Application Priority Data
Jun. 18, 2010 (KR) 10-2010-0058068

(51) Int. Cl. A61K 36/48

(52) U.S. Cl. CPC A61K 36/48
USPC 424/757

(57) ABSTRACT

The present invention relates to an antithrombotic composition containing a fraction of a colored-bean extract. The antithrombotic composition is highly useful for the treatment of diseases of the circulatory system, including cardiovascular disease, cerebrovascular disease, arteriosclerosis, hypertension and diabetes, which are caused by thrombosis.

### TECHNICAL FIELD

The present invention relates to an antithrombotic composition containing a fraction of a colored-bean extract as an active ingredient.

### BACKGROUND ART

In recent years, the development of diseases of the circulatory system in the middle-aged and elderly groups, which are called adult diseases, has increased due to a change in dietary lifestyle and an increase in intrinsic and extrinsic stress, and this increase has become a serious problem. According to statistics provided by the Korean National Statistical Office in 2008, cerebrovascular diseases and heart diseases, including atherosclerosis, cerebral hemorrhage, stroke and cerebral infarction, are the first or second leading cause of death among death causes except for cancer. The major cause of these diseases is thrombus, and thrombosis is a pathology caused by excessive platelet aggregation. When blood vessels are damaged, platelets are activated by agonists such as collagen, thrombin, ADP (adenosine diphosphate) and the like, resulting in platelet adhesion, secretion and aggregation. This process plays an important role not only in hemostasis, but also in the development of diseases of the circulatory system, including thrombosis.

Coronary artery disease, a disease of the circulatory system, is known as one of causes of death, which are most frequent worldwide. Particularly, acute coronary syndrome is a coronary artery disease that is the direct cause of death. Acute coronary syndrome can be divided into unstable angina, non-ST-segment elevation myocardial infarction (NSTE; non Q-wave MI), and ST-elevation myocardial infarction (STE MI; Q-wave MI), and the former two also are collectively referred to as NSTE ACS (non-ST-elevation acute coronary syndrome), because treatment and prognosis are similar between the two. In chronic stable angina, ischemia is caused by a decrease in the effective diameter of coronary arteries due to atherosclerosis, but acute coronary syndrome has a mechanism different from that of chronic stable angina. Acute coronary syndrome occurs when blood flow is rapidly reduced or blocked by intracoronary acute thrombosis caused by the rupture or erosion of an atherosclerotic plaque.

More specifically, in acute coronary syndrome, the intravascular thrombotic process is the same as the process in which hemostasis occurs after traumatic vascular injury. In other words, when the rupture or erosion of a vulnerable atherosclerotic plaque occurs, connective tissue below endothelial cells is exposed to blood. Platelets in blood adhere to the exposed connective tissue (platelet adhesion), and the platelets are activated by mechanical and biochemical stimulation to secrete TxA2 (thromboxane A2), ADP (adenosine diphosphate), epinephrine and the like (platelet activation). These secreted substances activate GP IIb/IIIa receptor on the platelet surface, and the activated GP IIb/IIIa receptor causes platelets to aggregate by fibrinogen (platelet aggregation). Platelets are activated by many pathways, but are finally aggregated by fibrinogen through GP IIb/IIIa receptor, and thus this process is the final common pathway of platelet aggregation. Primarily produced thrombus is “white thrombus” rich in platelets and corresponds to the primary hemostasis of a hemostasis process.

The produced PRT (platelet-rich thrombus) is produced on the vascular wall of the injury portion of atherosclerotic plaques and usually does not completely occlude coronary arteries (Mural thrombus). This clinically corresponds to NTSE ACS. On PRT (platelet-rich thrombus), thrombin (factor Ha) is produced by activation of coagulation and chain reactions. Thrombin activates fibrinogen to fibrin, and activated fibrin forms a meshwork while a thrombus containing blood cells such as red blood cells is formed. The formed thrombus is “red thrombus” and corresponds to the secondary hemostasis of the hemostasis process. Thrombosis/thrombolysis balance is continued toward thrombolysis production, blood vessels are completely occluded, resulting in STE MI.

Antithrombotic drugs can be divided into anti-platelet agents for inhibiting platelets which are involved in primary hemostasis, and anticoagulants for inhibiting coagulation in secondary hemostasis. Anti-platelet agents include aspirin which inhibits TxA2 production, clopidogrel and ticlopidine, which are ADP receptor blockers, abciximab that is a GP IIb/IIIa receptor blocker, and the like. In addition, theopylline, molsidomine, verapamil, nifedipine, nitroglycerine and the like are known to promote of cAMP and cGMP, which inhibit the recruitment of Ca²⁺. Anticoagulants include heparin that activates antithrombin III to degrade thrombin, the direct thrombin inhibitor (DTI) hirudin, the vitamin K antagonist warfarin and so on.

However, the above-mentioned drugs can cause various side effects, including excessive inhibition of hemostasis in the human body, sterility, and disorders of the digestive system. Thus, there is a need to develop an agent, which significantly reduces the side effects of antithrombotic drugs and, at the same time, is safe.

### DISCLOSURE

### Technical Problem

One aspect of the present invention is to provide a composition having an antithrombotic effect

Another aspect of the present invention is to provide a pharmaceutical composition or health food composition having an antithrombotic effect

### Technical Solution

One aspect of the present invention provides a composition containing, as an active ingredient, a fraction of a colored-bean extract obtained using water, a C₁-C₅ alcohol or a mixture thereof.

### Advantageous Effects

A composition according to one aspect of the present invention has an excellent antithrombotic effect, comprises a natural substance having no side effect and can be variously used in the pharmaceutical and health food fields.

### DESCRIPTION OF DRAWINGS

FIG. 1 is a graph showing the platelet aggregation inhibitory effect as a function of the concentration of a 50% ethanol fraction obtained by fractionating a 20% ethanol extract of field beans with HP-20.

### MODE FOR INVENTION

Previous studies on beans were conducted in order to isolate and purify pharmacologically active ingredients from beans, and studies on the medical use of beans themselves are still insufficient. In addition, beans have been extracted using extraction methods employing high-concentration organic solvents, which are extraction methods commonly used to extract natural materials.

Bean extracts contain several unknown components in addition to identified components, and some of these components may exhibit pharmacological effects beneficial to the human body. In addition, useful components of beans can also not be extracted by an extraction method using a high-concentration organic solvent. Unlike extraction methods that are generally used to natural materials and medicinal herbs, the present inventors have obtained an extract from beans using water or a low-concentration organic solvent as an extraction solvent, and have found that a fraction obtained by fractionating the obtained bean extract with resin exhibits potent antithrombotic effects.

A composition according to one aspect of the present invention is characterized in that it contains a fraction of a bean extract obtained using water, an organic solvent or a mixture thereof as an extraction solvent. A composition according to another aspect of the present invention may be an antithrombotic composition containing a fraction of a colored-color extract obtained using water, a C₁-C₅ alcohol or a mixture thereof as an extraction solvent.

In one embodiment of the present invention, the organic solvent is not specifically limited and may be a C₁-C₅ lower alcohol. The C₁-C₅ lower alcohol may be, for example, any one or a mixture of two or more selected from the group consisting of methanol, ethanol, isopropanol, n-propyl alcohol, n-butanol and isobutanol. Specifically, the C₁-C₅ lower alcohol may be ethanol. In another embodiment of the present invention, the concentration of the C₁-C₅ alcohol is 1-70% (v/v), specifically 1-40% (v/v), more specifically 5-25% (v/v), and even more specifically 7-20% (v/v). For example, the solvent may be 5-25% (v/v) ethanol, and more specifically 10% or 20% (v/v) ethanol.

In the present invention, beans are extracted using water or a low-concentration lower alcohol. The present inventors have conducted various studies and repeated tests and, as a result, have found that a colored-bean extract obtained using a lower alcohol, for example, ethanol, particularly low-concentration ethanol, among a variety of organic solvents, is effective in improving blood circulation and preventing and treating obesity and diabetes and has excellent effects on the prevention and treatment of hyperlipidemia, thereby completing the present invention.

In one embodiment of the present invention, the colored-bean extract fraction may be a resin fraction of a water or lower alcohol extract, and specifically a HP-20 resin fraction. The resin fraction is, for example, a fraction obtained by fractionation using a synthetic adsorbent column including a polymer of benzene and polystyrene, and specifically a HP-20 resin fraction. The present inventors performed a test for extracting various fractions from a colored-bean extract obtained using low-concentration ethanol as a typical example. As a result, it was found that a fraction obtained by fractionating the extract with HP-20 resin has excellent antithrombotic effects. Particularly, it was shown that a HP-20 resin 50% ethanol fraction has a very excellent effect compared to a water fraction.

In one embodiment of the present invention, the colored-bean extract fraction may be a resin fraction of a water or lower alcohol extract of colored beans, and specifically a fraction obtained by fractionating a low-concentration ethanol extract with HP-20 resin.

As used herein, the term “colored bean” is meant to include beans whose husks have deep colors, including black, red, yellow and green. The colored bean may be any one or more selected from the group consisting of Seoritae (Glycine max MERR), Seomoktae (Rhynchosia Nolubilis), blue bean (Glycine max MERR), yellow bean (Glycine max MERR), field bean (Phaseolus vulgaris L.), kidney bean (Phaseolus vulgaris), pinto bean (Phaseolus vulgaris L.), small red bean (Vigna angularis), small black bean (Phaseolus angularis W. F. WIGHT.), sprouting bean (Glycine max (L.) Merr.), soybean (Glycine max), black soybean (Glycine max (L.) Merr.), mung beans (Vigna radiate) and Sun-bi bean (Glycine max (L) Merr.). In one embodiment of the present invention, the colored bean may be a field bean. The colored bean can be named in various manner ways depending on regions, classification, random talk and the like. As used herein, the term “colored-bean extract” refers collectively to substances extracted from colored beans by various extraction processes and includes, for example, substances extracted using water or an organic solvent. As used herein, the term “fraction” refers collectively to various fractions of any substance and includes, for example, fractions of extracts. More specifically, the term “fraction” includes a HP-20 resin fraction of the colored-bean extract.

A composition containing the colored-bean extract fraction according to the present invention has the effect of inhibiting platelet aggregation to inhibit thrombosis, and has the effect of inhibiting vasoconstriction to induce vascular relaxation. Thanks to such effects, the composition of the present invention is effective in improving blood circulation and can be effectively used for the prevention or treatment of obesity, diabetes and hyperlipidemia.

One aspect of the present invention provides a pharmaceutical composition comprising the above-described composition as an active ingredient. In one embodiment of the present invention, the pharmaceutical composition may be a composition for the prevention, alleviation or treatment of vascular diseases. The pharmaceutical composition comprising the composition according to the present invention has the effect of preventing thrombosis, inhibiting vasoconstriction and suppressing cholesterol. Specifically, the pharmaceutical composition may be a pharmaceutical composition for improving blood circulation by antithrombotic activity and may be a pharmaceutical composition for the alleviation or treatment of vascular diseases, including obesity, diabetes and hyperlipidemia. Examples of the vascular diseases include obesity, diabetes, stroke, cerebral hemorrhage, arteriosclerosis, angina, myocardial infarction, hypertension, anemia, migraine or hyperlipidemia.

When the composition according to the present invention is applied to medical drugs, it can be formulated in liquid, semi-solid or solid forms for oral or parenteral administration using conventional inorganic or inorganic carriers.

Examples of formulations for oral administration include tablets, pills, granules, soft or hard capsules, powders, fine granules, emulsions, syrups, pellets and the like. Examples for formulations for parenteral administration include injectable solutions, drops, ointments, lotions, sprays, suspensions, suppositories and the like. The active ingredient of the present invention can be easily formulated according to conventional methods, and surfactants, excipients, colorants, flavoring agents, preservatives, stabilizers, buffers, suspending agents or other conventional additives can be suitably used for the formulation.

The pharmaceutical composition according to the present invention may be administered orally, parenterally, intrarectally, topically, transdermally, intravenously, intramuscularly, intraperitoneally or subcutaneously.

In addition, the dose of the active ingredient will vary depending on various factors, including the age, sex and weight of the subject to be treated, the particular disease or physiological condition to be treated, the severity of the physiological condition, the route of administration, and the physician's judgment. The determination of the dose based on such factors is within the level of those skilled in the art. The active ingredient may generally be administered at a dose of 0.001 mg/kg/day to 2000 mg/kg/day, and specifically 0.5 mg/kg/day to 1500 mg/kg/day, but is not limited thereto.

One aspect of the present invention provides a food additive, functional food or health food composition comprising the composition of the present invention as an active ingredient. In one embodiment of the present invention, the food additive, functional food or health food composition comprising the composition of the present invention as an active ingredient may be a composition for improving blood circulation and may be a composition for preventing or alleviating vascular diseases, including obesity, diabetes or hyperlipidemia. Examples of the vascular diseases include obesity, diabetes, stroke, cerebral hemorrhage, arteriosclerosis, angina, myocardial infarction, hypertension, anemia, migraine or hyperlipidemia.

Examples of the food additive or functional food comprising the composition of the present invention include fermented milk, cheese, yogurt, juice, probiotics, health supplement foods, and so on.

In one embodiment of the present invention, the composition may contain other components which can improve the effects of the present invention, as long as such components do not impair the effects of the present invention. For example, the composition of the present invention may further comprise additives, including fragrance, pigments, bactericides, antioxidants, preservatives, moisturizing agents, thickeners, inorganic salts, emulsifiers and synthetic polymers.

In addition, the composition of the present invention may further comprise auxiliary components, including water-soluble vitamins, oil-soluble vitamins, polymer peptides, polysaccharides and seaweed extracts. The above components can be suitably selected without difficulty by those skilled in the art depending on the formulation or intended use of the composition and may be added in an amount selected within the range that does not impair the object and effect of the present invention. For example, the above components may be added in an amount of 0.01-5 wt %, and specifically 0.01-3 wt %, based on the total weight of the composition.

The composition of the present invention may be formulated in various forms, including solutions, emulsions, viscose mixtures, tablets, and powders, which can be administered by various methods, including sample drinking, injection, spraying or squeezing.

Hereinafter, the present invention will be described in further detail with reference to examples and test examples. It is to be understood, however, that these examples and test examples are for illustrative purposes and are not intended to limit the scope of the present invention.

### EXAMPLE 1

### Separation of 10% Ethanol Extract of Field Beans by HP-20 Resin

1 kg of dried field beans were immersed in 5 L of 10% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 10% ethanol extract sample.

Using the hydrophobic resin DIAION HP-20 (SUPELCO), a resin fraction was obtained from the 10% ethanol extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 10% ethanol extract of field beans was dissolved in water and added to the HP-20 resin-packed column, and 75 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 2

### Separation of 20% Ethanol Extract of Field Beans by HP-20 Resin

1 kg of dried field beans were immersed in 5 L of 20% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 20% ethanol extract sample. Using the hydrophobic resin DIAION HP-20 (SUPELCO), a resin fraction was obtained from the 20% ethanol extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 20% ethanol extract of field beans was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 3

### Separation of 30% Ethanol Extract of Field Beans by HP-20 Resin

1 kg of dried field beans were immersed in 5 L of 30% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 30% ethanol extract sample.

Using the hydrophobic resin DIAION HP-20 (SUPELCO), a resin fraction was obtained from the 30% ethanol extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 30% ethanol extract of field beans was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 4

### Separation of 40% Ethanol Extract of Field Beans by HP-20 Resin

1 kg of dried field beans were immersed in 5 L of 40% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 40% ethanol extract sample.

Using the hydrophobic resin DIAION (SUPELCO), a resin fraction was obtained from the 40% ethanol extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 40% ethanol extract of field beans was dissolved in water and added to the HP-20 resin-packed column, and 750 ml of distilled water, 750 ml of 50% ethanol and 500 ml of ethanol were sequentially introduced into the column while each of the solvents from the column was received in 250-ml Erlenmeyer flasks. Each of the discharged solvents was concentrated under reduced pressure and freeze-dried, thereby obtaining fraction samples.

### EXAMPLE 5

### Separation of 70% Ethanol Extract of Field Beans by HP-20 Resin

1 kg of dried field beans were immersed in 5 L of 70% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 70% ethanol extract sample.

Using the hydrophobic resin DIAION HP-20 (SUPELCO), a resin fraction was obtained from the 70% ethanol extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 70% ethanol extract of field beans was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 6

### Separation of Water Extract of Field Beans by HP-20 Resin

1 kg of dried field beans were immersed in 5 L of water and extracted under reflux at 60° C. for 3 hours, and the extract was allowed at room temperature for a predetermined time. Then, the extract was filtered, concentrated under reduced pressure, and freeze-dried, thereby preparing a water extract sample.

Using the hydrophobic resin DIAION HP-20 (SUPELCO), a resin fraction was obtained from the water extract of field beans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 of the water extract of field beans was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 7

### Separation of Extract of Seoritae by HP-20 Resin

1 kg of dried Seoritae were immersed in 5 L of 20% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 20% ethanol extract sample.

Using the hydrophobic resin DIAION HP-20 (SUPELCO), a resin fraction was obtained from the 20% ethanol extract of Seoritae in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 20% ethanol extract of Seoritae was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 8

### Separation of Extract of Soybeans by HP-20 Resin

1 kg of dried soybeans were immersed in 5 L of 20% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 20% ethanol extract sample.

Using the hydrophobic resin DIAION HP-20 (SUPELCO), a resin fraction was obtained from the 20% ethanol extract of soybeans in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 20% ethanol extract of soybeans was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### EXAMPLE 9

### Separation of extract of Seomoktae Beans by HP-20 Resin

1 kg of dried Seomoktae beans were immersed in 5 L of 20% ethanol solution and extracted under reflux at 60° C. for 3 hours, and the extract was allowed to stand at room temperature for a predetermined time. Then, the extract was filtered, concentrated under reduced pressure, and freeze-dried, thereby preparing a 20% ethanol extract sample.

Using the hydrophobic resin DIAION HP-20 (SUPELCO), a resin fraction was obtained from the 20% ethanol extract of Seoritae in the following manner.

For separation by HP-20 resin, HP-20 resin was packed into a column to a length of 30 cm, and then washed twice with 500 ml of ethanol, twice with 500 ml of 50% ethanol, and twice with 500 ml of distilled water. 6 g of the 20% ethanol extract of Seoritae was dissolved in water and added to the HP-20 resin-packed column, and 750 ml of distilled water, 750 ml of 50% ethanol and 500 ml of ethanol were sequentially introduced into the column while each of the solvents discharged from the column was received in 250-ml Erlenmeyer flasks. Each of the discharged solvents was concentrated under reduced pressure and freeze-dried, thereby obtaining fraction samples.

### TEST EXAMPLE 1

### Observation of Inhibition of Human Platelet Aggregation Induced by Collagen

In order to examine the change in activity of an extract with a change in the ethanol content of an extraction solvent, the following test was performed using field bean extracts obtained using extraction solvents having varying water or ethanol contents.

To separate human platelet-rich plasma (PRP), 3.2% sodium citrate was used as an anti-coagulating agent, and blood was collected from the veins of healthy men who have not taken any medicine for 2 weeks or more. 150 g of the collected blood was centrifuged for 15 minutes, the supernatant (PRP) was collected, and the residue was centrifuged to separate platelet-poor plasma (PPP). Platelets in the collected PRP were counted using a cell counter, and PRP was diluted with PPP to a concentration of 3×10⁸ platelets/ml and used in the test.

Platelet aggregation activity was determined based on the change in absorbance using a lumi-aggregometer (Chrono-Log Co., USA). PRP was pre-incubated in a thermomixer at 37° C. for 2 minutes, and a field bean extract was added to the pre-incubated PRP at a concentration of 200 μg/ml and incubated for 7 minutes. 500 μl of the incubated PRP was added to and incubated in a silicone-coated cuvette for platelet aggregation and incubated for 3 minutes. Then, collagen as a platelet aggregation inducer was added at the minimum concentration of 1-3 μg/ml, which cause the maximum platelet aggregation, after which the reaction was observed for 6 minutes. The results of the observation are expressed in Table 1 below as plate aggregation inhibitory rates (%) relative to the platelet aggregation inhibitory rate (0%) of the control group treated with collagen alone.

TABLE 1

| Sample | Inhibitory rate (%) |
|---|---|
| Water extract | 69.0 |
| 10% ethanol extract | 57.3 |
| 20% ethanol extract | 76.3 |
| 30% ethanol extract | 38.7 |
| 40% ethanol extract | 34.7 |
| 50% ethanol extract | 39.7 |
| 70% ethanol extract | 10.7 |

As can be seen in Table 1 above, the water, 10% and 20% ethanol extracts showed excellent effects on the inhibition of platelet aggregation compared to other extracts. In order to examine the activity of a HP-20 fraction of each of the above ethanol extracts, fractionation was performed with HP-20 resin as described in Examples 1 to 5.

### TEST EXAMPLE 2

### Platelet Aggregation Activities of HP-20 Fractions of the Ethanol Extracts Obtained Using Extraction Solvents having Varying Ethanol Contents

Using water fractions and 50% ethanol fractions of field bean extracts, obtained by fractionation with HP-20 resin as described in Examples 1 to 5, a test was performed in the same manner as Test Example 1. The results of the test are expressed in Table 2 below as platelet aggregation inhibitory rates (%).

TABLE 2

| | Before | | | |
|---|---|---|---|---|
| | fractionation with | Before fractionation with HP-20 resin | | |
| | HP-20 resin | Water fraction | 50% ethanol fraction | |
| | 200 μg/ml | 200 μg/ml | 100 μg/ml | 200 μg/ml |
| 10% ethanol | 57.3 | 8 | 81 | — |
| extract | | | | |
| 20% ethanol | 76.3 | 39.7 | 68 | 100 |
| extract | | | | |
| 30% ethanol | 38.7 | — | 29 | 62 |
| extract | | | | |
| 40% ethanol | 34.7 | 20 | 29 | 69 |
| extract | | | | |
| 70% ethanol | 10.7 | <5 | — | 67 |
| extract | | | | |

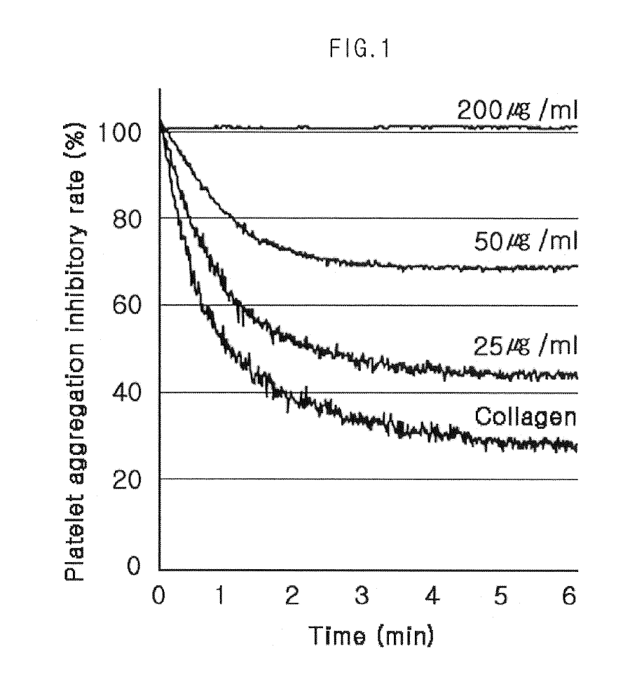

As can be seen in Tables 1 and 2 above, when the 30%, 40% and 70% ethanol extracts, which have showed relatively weak effects in Test Example 1, were fractionated with HP-20 resin, the 50% ethanol fraction of the obtained fractions showed an increased activity of inhibiting platelet aggregation. As can be seen in FIG. 1 showing the platelet aggregation inhibitory effect as a function of the 50% ethanol fraction obtained by fractionating the 20% ethanol extract of field beans with HP-20 resin, the platelet aggregation inhibitory effect of the 50% ethanol extract increased in proportion to the concentration thereof.

Thus, this suggests that fractionation with HP-20 concentrates platelet aggregation inhibitory activity on the 50% ethanol fraction.

### TEST EXAMPLE 3

### Platelet Aggregation Inhibitory Activity of Each HP-20 Fraction as a Function of the Kind of Bean

In order to evaluate the platelet aggregation inhibitory effects of various beans, the 20% ethanol extracts of various beans, which have showed the best effects on the inhibition of platelet aggregation, were fractionated with HP-20 resin according to the methods described in Examples 7 to 9.

Under the same conditions as those of Test Example 1, a test was performed using 200 μg/ml of each of a water fraction and 50% ethanol fraction of each bean extract, obtained by fractionation with HP-20 resin. The results of the test are expressed in Table 3 below as platelet aggregation inhibitory rates (%).

TABLE 3

| | Fractionation with | After fractionation with HP-20 resin | | |
|---|---|---|---|---|
| | HP-20 | Water fraction | 50% ethanol fraction | |
| | 200 μg/ml | 200 μg/ml | 100 μg/ml | 200 μg/ml |
| Soybeans | 19.3 | 4.3 | 66.0 | 91.3 |
| Seomoktae | 25.0 | 1.0 | 82.0 | 90.7 |
| Seoritae | 65.3 | 3.7 | 69.3 | 95.7 |
| Field beans | 76.3 | 39.7 | 93.0 | 100.0 |

As can be seen in Table 3 above, in the case of all kinds of beans, including field beans, the platelet aggregation inhibitory effects of the 50% ethanol fractions obtained by fractionation with HP-20 resin were significantly increased compared to those of the 20% ethanol extracts. This suggests that fractionation of the extracts with HP-20 is a major method for increasing platelet aggregation inhibitory activity, as described in Test Example 2. In addition, in the cases of not only the field bean and Seoritae extracts which have showed relatively high activities, but also the soybean extract which has showed relatively low activity, fractionation with HP-20 is a sure method for increasing the platelet aggregation inhibitory activities of the extracts.

### TEST EXAMPLE 4

### Observation of Specific Inhibition of Activity of Each Platelet Aggregation-Stimulating Source

In order to examine whether the HP-20 resin 50% ethanol fraction of the 20% ethanol extract of each bean, which has showed the highest inhibitory activity, specifically inhibits each of platelet aggregation-stimulating sources, the following test was carried out.

When a blood vessel is damaged, an endothelial cell layer in the damaged site is broken, the blood vessel is exposed to collagen, and ADP (adenosine diphosphate) and thrombin are secreted from platelets. These substances are known to stimulate platelet aggregation. Thus, in order to examine whether the 50% ethanol fractions obtained by fractionation with HP-20 resin to specifically inhibit platelet aggregation activities caused by ADP and thrombin, a test was performed in the same manner as Test Example, except that ADP and thrombin stimulations were applied instead of collagen stimulation. The results of the test are expressed in Table 4 below as platelet aggregation inhibitory rates (%).

TABLE 4

| | ADP | Thrombin |
|---|---|---|
| Soybeans | 75.0 | 45.0 |
| Seomoktae beans | 83.5 | 58.0 |
| Seoritae beans | 74.5 | 49.7 |
| Field beans | 99.0 | 57.0 |

As can be seen in Table 4, the HP-20 resin 50% ethanol fractions showed inhibitory effects not only on platelet aggregation induced by collagen, but also on platelet aggregation induced by other stimulants such as ADP and thrombin.

### TEST EXAMPLE 5

### Observation of Inhibitory Effect of HP-20 50% Ethanol Fraction of Each Bean Extract on Secretion of Activator Substance After Platelet Aggregation

In order to examine the inhibitory effect of the HP-20 resin 50% ethanol fraction of each bean extract on the production of thromboxane after platelet aggregation, 400 μg/ml of each fraction sample was added to PRP obtained in Test Example 1 and was incubated at 37° C. for 10 minutes, after which 5 μg/ml of collagen was added thereto and allowed to react for 6 minutes. 2 mM EDTA and 50 μM indomethacin were added thereto to stop the reaction. The resulting solution was centrifuged at 2000×g for 20 minutes, and the supernatant was taken. The amount of thromboxane in the supernatant was quantified by an enzyme immunoassay. The results of the test are expressed in Table 5 below as thromboxane production inhibitory rates (%).

TABLE 5

| | Inhibitory rate (%) |
|---|---|
| Soybeans | 57.6 |
| Seomoktae beans | 54.1 |
| Seoritae beans | 47.9 |
| Field beans | 74.4 |

As can be seen in Table 5 above, it was observed that the BP-20 resin 50% ethanol fractions inhibited the production of thromboxane after platelet aggregation.

### TEST EXAMPLE 6

### Observation of Inhibitory Effects on Aggregation of Platelets Isolated from SD Rats

In order to examine whether the HP-20 resin 50% ethanol fraction of the field bean extract has an inhibitory effect on the aggregation of platelets isolated from rat, a test was performed using 15 μg/ml of collagen and 10 μM of ADP in the same manner as Test Example 1. The fraction sample was used at a concentration of 200 μg/ml. The results of the test are expressed in Table 6 below as platelet aggregation inhibitory rates (%).

TABLE 6

| | Collagen | ADP |
|---|---|---|
| Field bean extract fraction | 83 | 69 |

As can be seen in Table 6 below, the HP-20 resin 50% ethanol fraction of the field bean extract has high inhibitory effects on platelet aggregation not only in humans but also in rats.

1. A method for inhibiting thrombosis, the method comprising administering an effective amount of a resin fraction of a colored-bean extract obtained by extracting with water, a C₁-C₅ alcohol or a mixture thereof to a subject in such need.

2. The method of claim 1, wherein the concentration of the C₁-C₅ alcohol is 1-40%(v/v).

3. The method of claim 1, wherein the C₁-C₅ alcohol includes any one or more selected from the group consisting of methanol, ethanol, isopropanol, n-propyl alcohol, n-butanol and isobutanol.

4. The method of claim 1, wherein the colored bean is one or more selected from the group consisting of Seoritae, Seomoktae, blue bean, yellow bean, field bean, kidney bean, pinto bean, small red bean, small black bean, sprouting bean, soybean and black soybean.

5. The method of claim 1, wherein the resin fraction is a fraction obtained by fractionation using a synthetic adsorbent column including a polymer of benzene and polystyrene.

6. The method, of claim 1, wherein the C₁-C₅ alcohol is a 5-25% (v/v) ethanol.

7. The method of claim 1, wherein the resin fraction of the colored-bean extract is a fraction by fractionating an ethanol extract of colored beans using a synthetic adsorbent column including a polymer of benzene and polystyrene.

8. A method for alleviating or treating vascular disease, the method comprising administering an effective amount of a resin fraction of a colored-bean extract by extracting with using water, a C1-C5 alcohol or a mixture thereof to a subject in such need.

9. The method of claim 8, wherein the vascular disease is obesity, diabetes, stroke, cerebral hemorrhage, arteriosclerosis, angina, myocardial infarction, hypertension, anemia, migraine or hyperlipidernia.

10. (canceled)

11. (canceled)
